(19) United States
(12) Patent Application Publication
(10) Pub. No.: US 2003/0225388 A1
(43) Pub. Date: Dec. 4, 2003

(54) Absorbent interlabial device

(76) Inventor: Ekavira Bhavani, Chennai (IN)

Correspondence Address: LAW OFFICES OF NAREN CHAGANTI, 432 S. CURSON AVE, STE. 12H, LOS ANGELES, CA, 90036, US

(21) Appl. No.: 10/156,510

(22) Filed: May 28, 2002

(51) Int. Cl. A61F 13/15; A61F 13/20

(52) U.S. Cl. 604/385.17; 604/367; 604/385.03

(57) ABSTRACT

The present invention relates to an absorbent article designed and configured to fit between the labia. The article employs a plurality of stacked, layered elements and provides for collection of bodily fluids and solids typically left unabsorbed by conventional interlabial products. The material used for absorbent purposes includes gum karaya, a plant extract, sometimes in combination with undyed compressed cotton and/or cellulose. Further embodiments include acrylic polymer to enhance the absorbent properties of gum karaya.

### TECHNICAL FIELD

This invention relates to absorbent devices, and more particularly to an absorbent device that is worn interlabially by, for example, female wearers for catamenial purposes, incontinence protection, or both.

### BACKGROUND

All manner and variety of absorbent articles configured for the absorption of bio fluids excreted from a human body such as menses, urine and feces are, of course, well known. There are two basic types of feminine protection devices: sanitary napkins, which have been developed for external wear about the pudendal region, and tampons, which have been developed for internal wear within the vaginal cavity for interruption of menstrual flow therefrom.

The term “labia” as used herein refers generally to both the labia majora and labia minora. The labia terminate anteriorly and posteriorly at the anterior commissure and the posterior commissure, respectively. It will be recognized by those skilled in the art that there is a wide range of variation among women with respect to the relative size and shape of labia majora and labia minora. For purposes of the present description, however, such differences need not be specifically addressed. It will be recognized that the disposition of the absorbent interlabial device into the interlabial space of a wearer as defined above will require placement between the inside surfaces of the labia majora without regard to the precise location of the boundary between the labia majora and the labia minora for a particular wearer. For a more detailed description of this portion of the female anatomy, attention is directed to Gray's Anatomy, Running Press 1901 Ed. (1974), at 1025-1027.

The term “interlabial space” refers to that space in the pudendal region of the female anatomy which is located between the inside surfaces of the labia majora extending into the vestibule. Located within this interlabial space are the labia minor, the vestibule and the principal urogenital members including the clitoris, the orifice of the urethra, and the orifice of the vagina. Standard medical authorities teach that the vestibule refers to the space bounded laterally by the inside surfaces of the labia minora and extending interiorly to the floor between the clitoris and the orifice of the vagina. Therefore, it will be recognized that the interlabial space as defined above may refer to the space between the inside surfaces of the labia majora, including the space between the inside surfaces of the labia minora also known as the vestibule. The interlabial space for purposes of the present description does not extend substantially beyond the orifice of the vagina into the vaginal interior.

As used herein the term “absorbent interlabial device” refers to a structure that has at least some absorbent components, and is specifically configured to reside at least partially within the interlabial space of a female wearer during use. Interlabial pads have the potential to provide freedom from inconvenience because of their small size and reduced risk of leakage. Such interlabial pads are colloquially referred to as “sanitary pads.”

Absorbent interlabial devices that are currently available have drawbacks associated with performance, comfort, or both. In tropical countries such as India, the commercially available interlabial pads are found to have insufficient absorbent properties. Accordingly there is a need for an improvement in the art.

### SUMMARY

The present disclosure is directed toward an absorbent interlabial device to absorb bio fluids such as menses, urine and feces. The disclosed device may be used in medical applications, such as in wound dressings, where other bio fluids such as blood, sweat and other secretions of a human are to be absorbed.

In one embodiment, the disclosed absorbent interlabial device includes at least one layer of material containing an absorbent material combined with a fibrous material. The absorbent material may be, in an embodiment, gum karaya. The fibrous material may be compressed cotton or compressed cellulose.

In another embodiment, there are at least three layers of material, including an innermost layer; at least one middle layer and an outermost layer. The innermost layer is one that contacts the wearer's body being made substantially of undyed thin knitted cotton fabric. Alternative embodiments may contain, instead of the undyed thin knitted cotton fabric, a layer of ultra thin micro perforated film or a combination of both the undyed thin knitted cotton fabric and the ultra thin micro perforated film.

The at least one middle layer is configured to contain a composite material including compressed cotton mixed with an absorbent material. Alternative embodiments may use, instead of compressed cotton, compressed cellulose. Further alternative embodiments may contain a combination of both compressed cotton and compressed cellulose. The absorbent material may be, in an embodiment, gum karaya powder. In other embodiments, the absorbent material may be acrylic polymer powder.

Alternative embodiments of the absorbent interlabial device may be designed in such a way as to provide extra cushion in the area that makes contact with sensitive body parts. For example, a portion that is closest to the wearer's private parts may be shaped to the contours requirement of the private parts.

Further designs are disclosed to accord the wearer a secure feeling. In an embodiment, the disclosed device may contain wings (or flaps) designed to overlap one over the other to contain the fluids within the device. Additionally, the device may be designed to maintain in tact with the inner wear. Finally, the device may, in alternative embodiments, be designed to provide some absorbent material in the wings (or flaps).

### BRIEF DESCRIPTION OF THE DRAWINGS

These and other objects, features and advantages of the presently disclosed device may be more readily understood from the following detailed description with reference to the accompanying drawings, wherein like numerals refer to like parts, and wherein

FIG. 1 is a side view of an illustrative multi-layered absorbent interlabial device;

FIG. 2 is a top view of the illustrative multi-layered absorbent interlabial device of FIG. 1;

FIG. 3 is a top view of a first middle layer of a multi-layered absorbent interlabial device; and

FIG. 4 is a view of a multi-layered absorbent interlabial device with folded wings.

### DETAILED DESCRIPTION

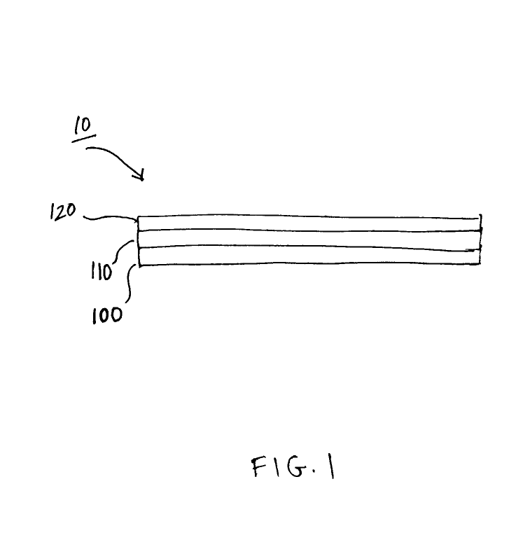

FIG. 1 shows an illustrative absorbent interlabial device 10 designed according to the principles disclosed herein. Note that the illustrative embodiments described herein to elaborate and elucidate the instant inventive principles should not be viewed as limitations. FIG. 1 shows that the absorbent interlabial device 10 may contain three layers of material, layers 100, 110, and 120. Layer 120 is the layer that could be in closest contact with a human wearer of the absorbent interlabial device. The layers described herein are layers of material, the properties of which will be described in detail below. It is, of course, understood that the layers of material may be configured to contain a specified thickness depending on a particular embodiment. The layers are stacked over one another and each layer in such a stack may be of equal or different dimensions with respect to other layers in the stack. It should further be noted that the materials comprising the layers are flexible thereby providing some comfort to a wearer.

Layer 100 could be the layer furthest from layer 120. For convenience, one may label layer 100 as the “lower” or the “outer” layer; layer 110 as the “middle” layer; and layer 120 as the “inner” or the “upper” layer. These designations and/or labels should not limit the inventive principles as the designations and/or labels are provided only to explain the principles. For example, the layer 110—the “middle” layer—may contain multiple layers of different materials, or it may contain a single layer of materials.

In an embodiment, layers 100, 110, 120 completely overlap each other. In other embodiments, the layers 100, 110 and 120 may not completely overlap each other. For example, in an embodiment, the layer 110 may contain certain material that does not completely overlap an upper and a lower layer.

The shape of the interlabial device may be substantially rectangular, substantially oval or substantially rectangular with substantially circular edges. Wings or flaps may be formed in alternative embodiments but these are not necessary in other embodiments. Other embodiments may have conical or other similar shapes for the device.

Each layer of material is constructed, in alternative embodiments, of varying dimensions. In an illustrative embodiment, each of the several ones of the middle layers is of 0.1 to 4 millimeters in thickness. Similarly, the outer layer may be constructed with a thickness of 0.1 to 4 millimeters; and the inner layer may be constructed to have a thickness of 0.1 to 4 millimeters.

### Method of Making

The layered interlabial article 10 can be formed as a single unit, such as in a foam molding operation, or the flexible elements can be manufactured individually and then attached to one another, such as by the use of adhesives, heat sealing with thermoplastic materials, hook and fastener technology, and ultrasonics. In the case of foam molding, by manipulating the foam material and/or molding process, both open celled and closed celled surfaces may be formed. Open celled surfaces are useful for absorbing bodily fluids, while closed celled surfaces can act as a barrier to prevent captured fluid from traveling completely through the article and onto a wearer's skin or clothing.

Additional methods and apparatuses useful in forming the articles of the present invention as a single structure are described in U.S. Pat. Nos. 5,165,152; 4,863,450; 4,859,273; 4,816,100; and 4,453,296. These methods generally consist of folding or rolling a web comprising fibrous materials onto itself into a blank, and then compressing the blank into a final form with heat and pressure. The blank typically takes a desired final form due to either the flowing and resetting of thermoplastic polymers in the web, or by the web being plastically deformed (non-recoverable deformation) sufficiently.

A useful web for this purpose is a single nonwoven web comprising blends of bicomponent thermoplastic fibers, such as polyethylene/polyester sheath core fibers, woodpulp, and superabsorbent polymers. The web may comprise several layers with the outer layers void of any absorbent material (i.e., woodpulp and superabsorbent polymer).

Construction adhesives known in the art may be optionally employed to aid in keeping the folds or pleats in their desired final form, or alternatively to adhere separately made flexible elements of the article together. A representative, non-limiting list of construction adhesives includes acrylics; starch based hot melts; adhesives based on block copolymers of vinyl aromatic hydrocarbon and one or more conjugated diene or hydrogenated aliphatic blocks; polylactic acids; hot melts based on polyolefins such as amorphous polyalphaolefins that may consist of one or more of the following monomers: propylene, ethylene, butane, and hexene; hot melts based on low density polyethylene or low density polyethylene copolymers including ethylene vinyl acetate, methyl acrylate, n-butyl acrylate, and acrylic acid.

Conventional methods used in constructing articles for managing bodily-fluids, such as sanitary napkins may also be used to make the interlabial article. These methods typically bond or encase multiple material layers or particulates together. The material layers or particulates may be in the form of apertured or non-apertured polymeric films, foam sheets, fibrous webs, superabsorbent fibers and/or particles, and woodpulp.

More particularly, in an embodiment, each of the three layers, 100, 110 and 120 may be individually formed and thereafter laid on top of each other in a press and pressed together. Alternatively, a sequential industrial process may prepare the materials in stages and assemble the entirety of the absorbent interlabial device 10 as a single device. In an embodiment, the three layers 100, 110 and 120 are attached at least at the edges of the layers. Such attachment may be made, for example, by pressing the layers 100, 110 and 120 together and/or by way of a number methods such as stitching the layers together, applying any known adhesive material to aid attachment of the layers to one another, and the like. This configuration allows a wearer to readily use the absorbent interlabial device thus formed.

### Materials Used In The Layered Interlabial Device

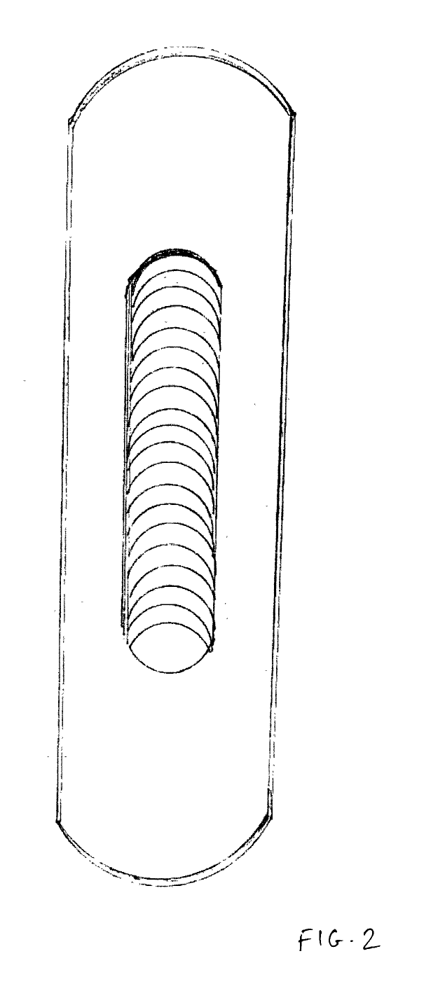

FIG. 2 depicts a top-view of a multi-layered absorbent interlabial device of FIG. 1. The top view advantageously depicts the inner layer 120, the layer closest to the body of the human wearer, of the absorbent interlabial device 10. All materials used to make the instant device are properly cleansed, sanitized to be of medical-grade, and are prepared to be free of infectious agents.

### The Body Contact Layer

In accordance with an embodiment, the inner or body-contact layer is made of undyed knitted cotton fabric. It has been discovered that knitted cotton fabric has certain benefits over nonwoven fabric in that the latter may cause allergic reactions in some wearers. Further, knitted cotton has good bio-fluid-absorbent property and is found to be useful in tropical countries such as India. Undyed knitted cotton fabric is non-allergic to most persons in view that there are no chemicals in that material. Accordingly, this too appears to be suitable because of its absorbent and fluid-retention properties. Alternative embodiments use micro perforated film designed to allow semi-permeability of bio fluids (i.e., allowing an easy flow of bio fluids in a first direction but not in a second (reverse) direction).

### A Middle Layer

The at least one middle layer is configured to contain a composite material including a fibrous material mixed with an absorbent material. Alternative embodiments may use, as the fibrous material, compressed cotton, compressed cellulose and other similar materials. Further alternative embodiments may contain a combination of both compressed cotton and compressed cellulose.

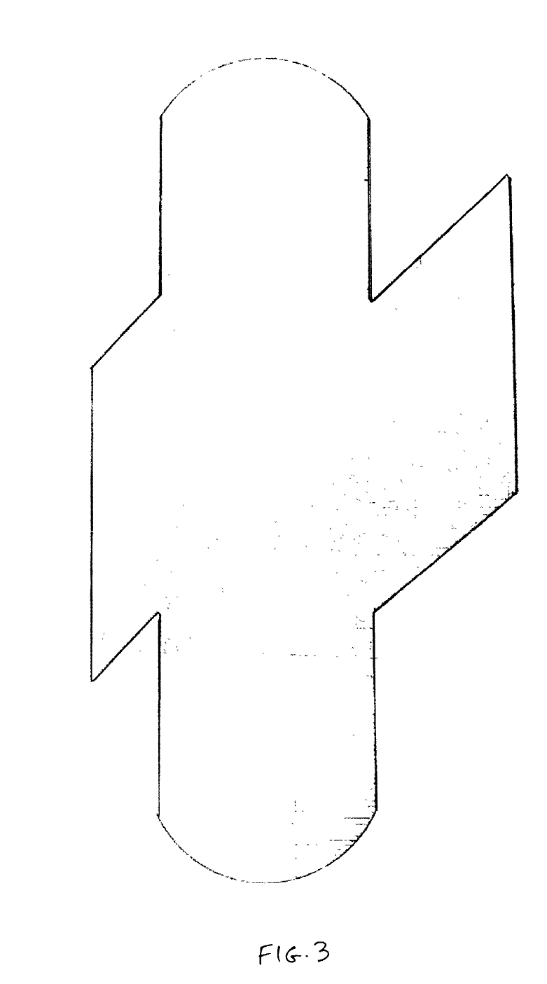

Shown in FIG. 3 is an illustrative middle layer constructed in accordance with the disclosed principles. It should be noted that one or more middle layers are possible. Other embodiments may use only two or fewer layers of materials but the principles of the invention apply equally well to such embodiments as well.

The absorbent material may be, in an embodiment, gum karaya powder. It has been discovered that compressed cotton (or in alternative embodiments, compressed cellulose) mixed with gum karaya and/or acrylic polymer has acceptable levels of absorption of bio fluids such as menstrual discharges, urine, and blood. Further, this composite material is found to retain bio fluids well. Acrylic polymer has the capacity absorb about 150 times its own weight of fluid, and about 500 times its own volume. Its absorbency performance against pressure is also acceptable in a confined space. Once the fluids are absorbed, a significant amount of the fluids is retained, minimizing leakage. Gum karaya has similar fluid absorbency and retention characteristics to acrylic polymer.

In other embodiments, the absorbent material may be acrylic polymer powder. The amount of gum karaya contained by the article can range from about 0.1 to about 7 grams, and more particularly from about 2 to about 6 grams.

More details of the material gum karaya are provided below.

### Gum Karaya (Sterculia Gum)

Gum karaya, sometimes known as Sterculia gum, is the dried exudation of the Sterculia Urens tree and other species of Sterculia. The tree is native to India.

Physical Characteristics

The highest-grade sorts of gum karaya are white, translucent and almost free of bark. The lower grades vary from light yellow to brown and may contain as much as 3% of insoluble impurities. Powdered gum karaya is white to greyish white.

Solubility

Gum karaya does not dissolve in water to give a clear solution but rather forms a colloidal solution. Powdered gum karaya swells in cold water to an extent that a 3% to 4% solution will produce a heavy gel of uniform smoothness and texture. Gum karaya forms viscous solutions in hydroalcoholic solutions ranging up to 60% to 35% alcohol concentration.

Viscosity

The viscosity of gum karaya is largely dependent on its freshness, that is, how recently it was gathered from the trees. The viscosity is affected by conditions of climate and growth. Viscosity is also affected by storage. Powdered gum karaya shows a decrease in viscosity after storing over 6 months. Gum karaya sols are sensitive to alkali and attain their maximum viscocity at pH 8.5. Above that pH the sols tend to become stringy.

Chemical Characteristics

Gum karaya is a complex polysaccharide of high molecular weight. A molecular weight as high as 9,500,000 has been reported. On hydrolysis it yields galactose, rhamnose and galacturonic acid. Gum karaya occurs as a partially acetylated derivative. The acid number has been found to vary from 13.4 to 22.7. The variation in acid number is influenced not only by the source of the sample but also by its age. The gum has a peculiar property of splitting off free acetic acid and this loss is loosely correlated with the particle size. Trimethylamine has also been identified in the hydrolysis products. Gum karaya contains 12% to 14% moisture and less than 1% acid insoluble ash.

pH

The pH of a 1% Gum karaya solution is 4.6. If small amounts of alkali are added to change the pH to 7 or 8, the gum tends to have a buffering action effect and will gradually reduce the pH again to the acid size.

Compatibility

Gum karaya is compatible with other plant hydrocolloids as well as proteins and carbohydrates. There is apparently an incompatibility of Gum karaya gels with pyrilamine maleate, a strong hydrotrope and antihistaminic. Electrolytes also cause a viscosity drop well as excessive acid. Alkalis cause the gels to become stringy.

### The Outermost Layer

The outermost layer is advantageously made of nonwoven material made with a spunbond-meltblown-spunbond (SMS) process. Tests have shown that the SMS nonwoven fabric is substantially biodegradable. Moreover, this fabric may absorb a good amount of bio fluids in a first direction and in a second direction it may impede the flow of such fluids. It has been further observed that in the second direction the SMS nonwoven fabric does not allow a significant amount of blood and other bodily fluids to seep through, thereby mitigating the problem of stains on that direction. Moreover, the SMS nonwoven fabric is air-permeable, making it comfortable even as it acts as a barrier to air-borne bacteria. This combination of properties makes the SMS nonwoven fabric useful as an outermost layer, wherein the fluid absorbent first direction is advantageously configured to face the innermost layer, thereby exposing the second direction to the elements or clothes worn over an absorbent interlabial device. The principles of the instantly disclosed absorbent interlabial device may be adapted to function as a tampon, which is typically inserted into the vaginal cavity to absorb menstrual discharges therefrom.

Alternative embodiments of the absorbent interlabial device may be designed in such a way as to provide extra cushion in the area that makes contact with sensitive body parts. For example, a portion that is closest to the wearer's private parts may be shaped to the contours requirement of the human genitals or other private parts. Further designs are disclosed to accord the wearer a secure feeling. In an embodiment, the disclosed device may contain wings (or flaps) designed to overlap one over the other to contain the fluids within the device. Additionally, the device may be designed to maintain in tact with the inner wear. Finally, the device may, in alternative embodiments, be designed to provide some absorbent material in the wings (or flaps).

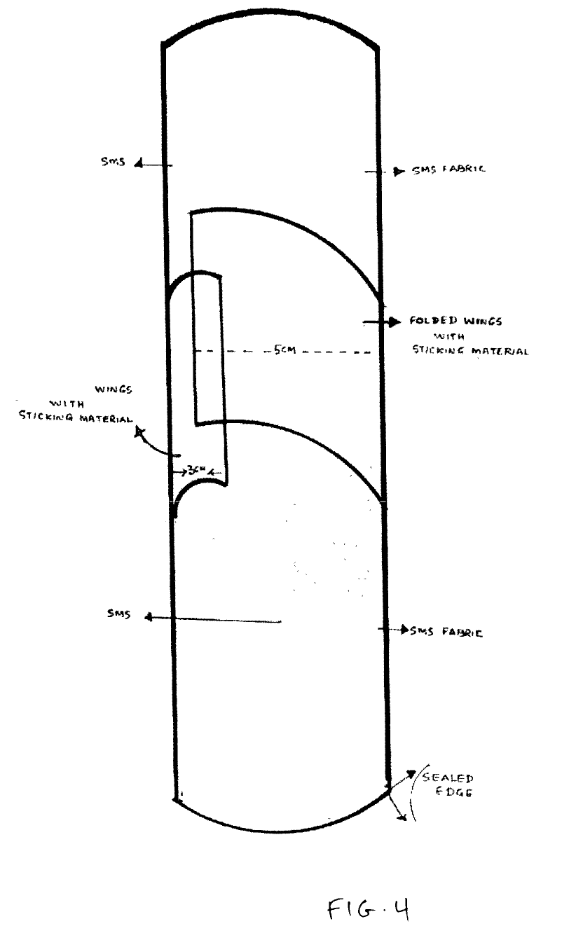

Shown in FIG. 4 is an embodiment of the absorbent interlabial device with flaps. The flaps may be coated with adhesive material to enable easy folding. The dimensions of the flaps are provided in the illustration as an example and not as a limitation of the principles disclosed.

The foregoing illustrates the principles and manufacture of an absorbent interlabial device. Persons of ordinary skill in the art may make modifications and/or rearrangements without departing from the spirit and scope of the principles disclosed herein. All such modifications and/or rearrangements should be considered to be within the scope of the appended claims.

What is claimed is:

1. An article of manufacture, comprising:
a layered absorbent interlabial article sized and configured to fit between a wearer's labia, said layered absorbent interlabial article comprising:
a body-contact layer, said body-contact layer adapted to be the layer closest to the wearer's body;
an outer layer; and
at least one middle layer, said at least one middle layer comprising fibrous material mixed with absorbent material.

2. The article of claim 1, wherein each of the body-contact layer, the at least one middle layer and the outer layer is made of a flexible material.

3. The article of claim 1, wherein the body-contact layer, the at least one middle layer and the outer layer are superimposed over each other.

4. The article of claim 1, wherein the body-contact layer, the at least one middle layer and the outer layer are bonded together to form a single unit.

5. The article of claim 1, wherein the body-contact layer comprises undyed knitted cotton fabric.

6. The article of claim 1, wherein the body-contact layer comprises semi-permeable micro-perforated film.

7. The article of claim 1, wherein the fibrous material comprises compressed cotton.

8. The article of claim 1, wherein the fibrous material comprises compressed cellulose.

9. The article of claim 1, wherein the absorbent material comprises gum karaya.

10. The article of claim 9, wherein the amount of gum karaya is between about 0.1 grams to about 7 grams.

11. The article of claim 9, wherein the amount of gum karaya is between about 2 to 6 grams.

12. The article of claim 1, wherein the absorbent material includes gum karaya mixed with acrylic polymer.

13. The article of claim 1, wherein each layer has a thickness of about 0.1 to about 4 millimeters.

14. The article of claim 1, wherein the outer layer comprises medical grade non-woven spunbond-meltblown-spunbond (SMS) fabric.

15. An article of manufacture, comprising:
a layered absorbent interlabial article sized and configured to fit between a wearer's labia, said layered absorbent interlabial article comprising:
at least one layer comprising fibrous material mixed with absorbent material.

16. The article of claim 15, further comprising a body-contact layer, said body-contact layer comprising undyed knitted cotton fabric.

17. The article of claim 15, further comprising a body-contact layer, said body-contact layer comprising semi-permeable micro-perforated film.

18. The article of claim 15, further comprising an outer layer, said outer layer comprises medical grade nonwoven spunbond-meltblown-spunbond (SMS) fabric.

19. The article of claim 15, wherein the fibrous material comprises compressed cotton.

20. The article of claim 15, wherein the fibrous material comprises compressed cellulose.

21. The article of claim 15, wherein the absorbent material comprises gum karaya.

22. The article of claim 21, wherein the amount of gum karaya is between about 0.1 grams to about 7 grams.

23. The article of claim 22, wherein the amount of gum karaya is between about 2 to 6 grams.

24. The article of claim 15, wherein the absorbent material includes gum karaya mixed with acrylic polymer.

25. The article of claim 15, wherein the layer has a thickness of about 0.1 to about 4 millimeters.
